(19) United States
(12) Patent Application Publication
(10) Pub. No.: US 2001/0051233 A1
(43) Pub. Date: Dec. 13, 2001

(54) 1-[2-(trans-4-alkyl cyclohexyl)ethyl]-4-aryl cyclohex-1-ene and liquid crystal composition containing the same

(76) Inventors: Dong-Jin Jeong, Seoul (KR); Kee-Han Uh, Yongin-city (KR); Vladimir S. Bezborodov, Minsk (BY); Valeri I. Lapanik, Minsk (BY); Genadz M. Sasnouski, Minsk (BY)

Correspondence Address: HOWREY SIMON ARNOLD & WHITE LLP, BOX 34, 1299 PENNSYLVANIA AVENUE NW, WASHINGTON, DC, 20004, US

(21) Appl. No.: 09/821,051

(22) Filed: Mar. 30, 2001

(30) Foreign Application Priority Data
Apr. 26, 2000 (KR) 00-22157

(51) Int. Cl. C09K 19/30; C07C 25/13

(52) U.S. Cl. 428/1.1; 252/299.63; 570/129

(57) ABSTRACT

A liquid crystal compound of 1-[2-(trans-4-alkyl cyclohexyl)ethyl]-4-aryl cyclohexyl-1-ene having the formula hereinbelow, and a liquid crystal composition containing the compound,

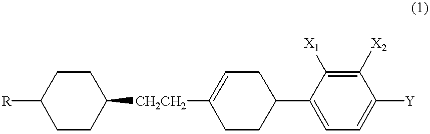

wherein R is CH₃(CH₂)ₙOₘ(CH₂)ₚ—; each of X₁ and X₂ is H, F or Cl; Y is F, Cl, —CN, —CF₃, —OCF₃ or an alkoxy group having 1 to 15 carbon atoms; each of n and p is an integer from 0 to 7; and m is 0 or 1. The liquid crystal composition has a much low nematic phase transition temperature and is characterized by a steep electro-optical curve, so that it reduces response time with a wide working temperature range.

### BACKGROUND OF THE INVENTION

1. Field of the Invention

The present invention relates to liquid crystal compounds, and more particularly, to 1-[2-(trans-4-alkyl cyclohexyl)ethyl]-4-aryl cyclohex-1-ene, and a liquid crystal composition containing the same.

2. Description of the Related Art

Liquid crystal display (LCDS) are based upon the electrical activity of organic compounds in response to light when the organic compounds exist in a liquid crystal phase. LCDs have a number of useful characteristics, including a compact size, light weight and low power consumption. Also, LCDs do not generate harmful electromagnetic waves. These advantages of LCDs have extended its applications covering desk-top computer monitors and television monitors as well as compact devices such as electronic calculators and notebook computers.

The performance of LCDs depends upon the physical and electro-optical characteristics of the liquid crystal composition. Thus, the composition of the liquid crystal mixture must be optimized using proper liquid crystal compounds having a particular physical property that may bring in proper electro-optical behavior.

Liquid crystal compounds are required to be chemically and thermally stable, and to be durable in an electric field. In addition, low viscosity, short addressing time, low threshold voltage and high contrast are required characteristics for liquid crystal compounds. Also, the liquid crystal compound must be present in a proper mesophase, for example, in a nematic or cholesteric phase at working temperatures. In addition, liquid crystals are used in a mixture form, rather than a pure form, and thus the mixing properties of each liquid crystal material, i.e., how easily the liquid crystals are mixed with other compounds, must be considered. Electrical conductivity, dielectric anisotropy, optical anisotropy and other physical properties must be considered according to the type of LCD cells and the application.

The present invention relates to 1,4-substituted cyclohex-1-ene series liquid crystal compounds among a variety of known liquid crystal compounds. The following identified patents: EP 198714, EP 410756, DE 4 203 719, U.S. Pat. No. 5,271,864, DE 3 906 040, DE 3 023 368 and EP 331091 teach of a variety of 1,4-substituted cyclohex-1-ene series liquid crystal compounds.

In particular, EP 198714 discloses 1,4-substituted cyclohex-1-ene series liquid crystal compounds having the following formula:

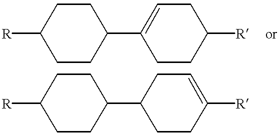

wherein each of R and R denotes a linear alkyl group having 1 to 9 carbon atoms, and each cyclohexane ring is arranged in a trans form.

U.S. Pat No. 5,271,864 discloses 1,4-substituted cyclohex-1-ene series liquid crystal compounds having the following formula:

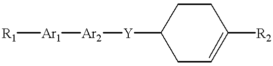

wherein each of R₁ and R₂, is selected from the group consisting of alkyl, cycloalkyl, alkenyl, alkoxy, thioalkyl and alkylsily groups having 1 to 20 carbon atoms; Y is —CH₂O—, —COO—, —OOC— or —OCH₂—; and each of Ar₁ and Ar₂ is selected from the group consisting of 1,4-phenyl, mono- or -difluorinated 1,4-phenyl, 2,5-pyrimidyl, 2,5-pyrazinyl, 2,5-thiadiazole, 3,6-pyridazinyl and trans-1,4-cyclohexyl, wherein at least one of Ar₁ and Ar₂ is a nitrogen-containing aromatic ring.

However, the disclosed 1,4-substituted cyclohex-1-ene series liquid crystal compounds have a relatively high phase transition temperature, and the ferroelectric or semi-ferroelectric phase thereof can be maintained within a narrow temperature range. Also, the electro-optical properties and dynamic parameters of the known liquid crystal compounds are unfavorable.

### SUMMARY OF THE INVENTION

To solve the above problems, it is an objective of the present invention to provide a 1,4-substituted cyclohex-1-ene series liquid crystal compound with improvement in terms of working temperature and electro-optical characteristics.

It is another objective of the present invention to provide a ferroelectric or semi-ferroelectric liquid crystal composition containing the 1,4-substituted cyclohex-1-ene series liquid crystal compound.

The present invention provides a liquid crystal compound of 1-[2-(trans-4-alkyl to cyclohexyl)ethyl]-4-aryl cyclohexyl-1-ene having the formula:

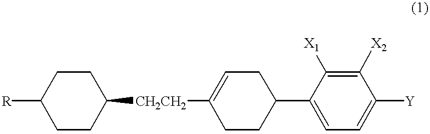

wherein R is CH₃(CH₂)ₙOₘ(CH₂)ₚ—; each of X₁ and X₂ is one of H, F and Cl; Y is F, Cl, —CN, —CF₃, —OCF₃ or an alkoxy group having 1 to 15 carbon atoms; each of n and p is an integer from 0 to 7; and m is 0 or 1.

### BRIEF DESCRIPTION OF THE DRAWING

The above objectives and advantages of the present invention will become more apparent by describing in detail preferred embodiments thereof with reference to the attached drawing in which:

FIG. 1 is the NMR spectrum of trans-4-(4-methoxy-2-fluorophenyl)-1-[2-(trans-4-butylcyclohexyl)ethyl cyclohex-1-ene obtained in Example 4.

### DETAILED DESCRIPTION OF THE INVENTION

The 1,4-substituted cyclohex-1-ene series liquid crystal compound having the formula (1) hereinabove is derived by the reactions expressed by the reaction scheme (1)

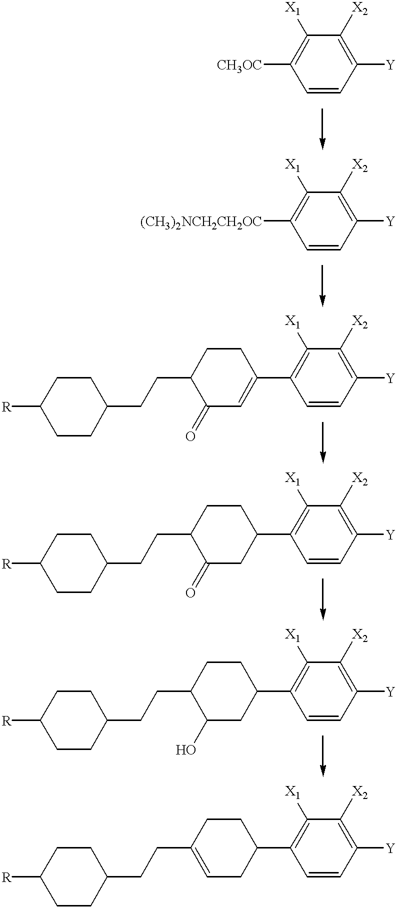

Hereinafter, a method for synthesizing the liquid crystal compound according to the present invention, and the results of performance tests with respect to liquid crystal compositions containing the liquid crystal compound will be described in greater detail in the following examples. However, these examples are merely illustrative and are not intended to limit the scope of the invention.

### EXAMPLE 1

### Synthesis of trans-4-(4-fluorophenyl)-1-[2-(trans-4-propyl cyclohexyl)ethyl]cyclohex-1-ene

Step 1—Preparation of Mannich salt from 4-fluoroacetophenone

1.0 mole of 4-fluoroacetophenone, 1.3 moles of paraformaldehyde, 1.3 mole of dimethylamine hydrochloride and 1 ml of concentrated hydrochroric acid were refluxed with 300 to 400 ml of ethanol (or isopropanol). After almost all of the solvent was evaporated, the resulting transparent solution was diluted with 0.1 liters of acetone, and allowed to sit in a refrigerator overnight. The resulting product was filtered, washed with cool acetone and dried to obtain Mannich salt with a yield of 68%.

Step 2—Preparation of (3-4-fluorophenyl)-6[2-(trans-4-propyl cyclohexyl)ethyl)cyclohex-2-ene-1-on

0.1 moles of the Mannich salt obtained in Step 1 was mixed with 0.1 moles of 2-[2-(trans-4-propylcyclohexyl)ethyl]acetoacetic acid ester, 0.3 moles of potassium hydroxide and 150 ml of dioxane, and refluxed for 5 hours while stirring. The resulting solution was cooled, treated with 10% sulfuric acid, and extracted with benzene. After removing the solvent from the resulting solution, the resulting product was crystallized with ethanol to obtain (3-4-fluorophenyl)-6[2-(trans-4-propyl cyclohexyl)ethyl)cyclohex-2-ene-1-on, with a yield of 65%.

Step 3—Preparation of trans-5-(4-fluorophenyl)-2[2-(trans-4-propyl cyclohexyl)ethyl)cyclohexaneon

12 grams of the (3-4-fluorophenyl)-6[2-(trans-4-propyl cyclohexyl)ethyl)cyclohex-2-ene-1-on obtained in Step 2 and 0.5 grams of potassium hydroxide were added to 50 ml of ethanol and tetrahydrofurane mixture (mixed in a 1:1 ratio by volume). The mixture was vigorously stirred at 30 to 40 C. at ordinary pressure in the presence of 0.3 to 10 % by weight carbonic palladium catalyst for hydrogenation until no more hydrogen was incorporated into the mixture. The resulting mixture was filtered to remove the catalyst used, and distilled to remove the solvent from the mixture. The obtained residues were dissolved in benzene. The resulting solution was washed with water and dried in the presence of anhydrous magnesium sulfate to evaporate the solvent. After two further crystallizations with ethanol, trans-5-(4-fluorophenyl)-2[2-(trans-4-propyl cyclohexyl)ethyl]cyclohexaneon was obtained, with a yield of 82%.

Step 4—Preparation of trans-5-(4-fluorophenyl)-2[2-(trans-4-propyl cyclohexyl)ethyl]cyclohexane-1-ol

6.5 grams of the trans-5-(4-fluorophenyl)-2[2-(trans-4-propyl cyclohexyl)ethyl]cyclohexaneon and 1.0 gram of sodium borohydride were added to 50 ml of isopropanol, and stirred at 40 C. for 4 to 5 hours. After cooling the resulting solution to room temperature, the cooled solution was decomposed with 10% hydrochroric acid to obtain a mixture of cyclohexanol isomers, and this process followed by the next step without performing further conversion or purification to the mixture.

Step 5—Preparation of trans-4-(4-fluorophenyl)-1-[2-(trans-4-propyl cyclohexyl)ethyl]cyclohex-1-ene

The alcohol isomer mixture obtained in Step 4 was dissolved in 25 ml of pyridine, and 2 ml of phosphorous oxychloride (POCl₃) was added to the mixture. The resulting mixture was refluxed for 5 hours and poured into water. The reaction product was extracted with ether and washed with water. After removing ether, the residue was dissolved in hexane and purified on a silica gel column flushed with pure hexane. After removing the solvent, the product was re-crystalized with isopropanol to obtain trans-4-(4-fluorophenyl)-1-[2-(trans-4-propyl cyclohexyl)ethyl]cyclohex-1-ene, with a yield of 61%.

### EXAMPLE 2

The same procedure of Example 1 was followed to synthesize trans4-(4-fluorophenyl)-1-[2-(trans-4-pentylcyclohexyl)ethyl cyclohex-1-ene, with a yield of 63%.

### EXAMPLE 3

The same procedure of Example 1 was followed to synthesize trans-4-(4,3-difluorophenyl)-1-[2-(trans-4-butylcyclohexyl)ethyl cyclohex-1-ene, with a yield of 58%.

### EXAMPLE 4

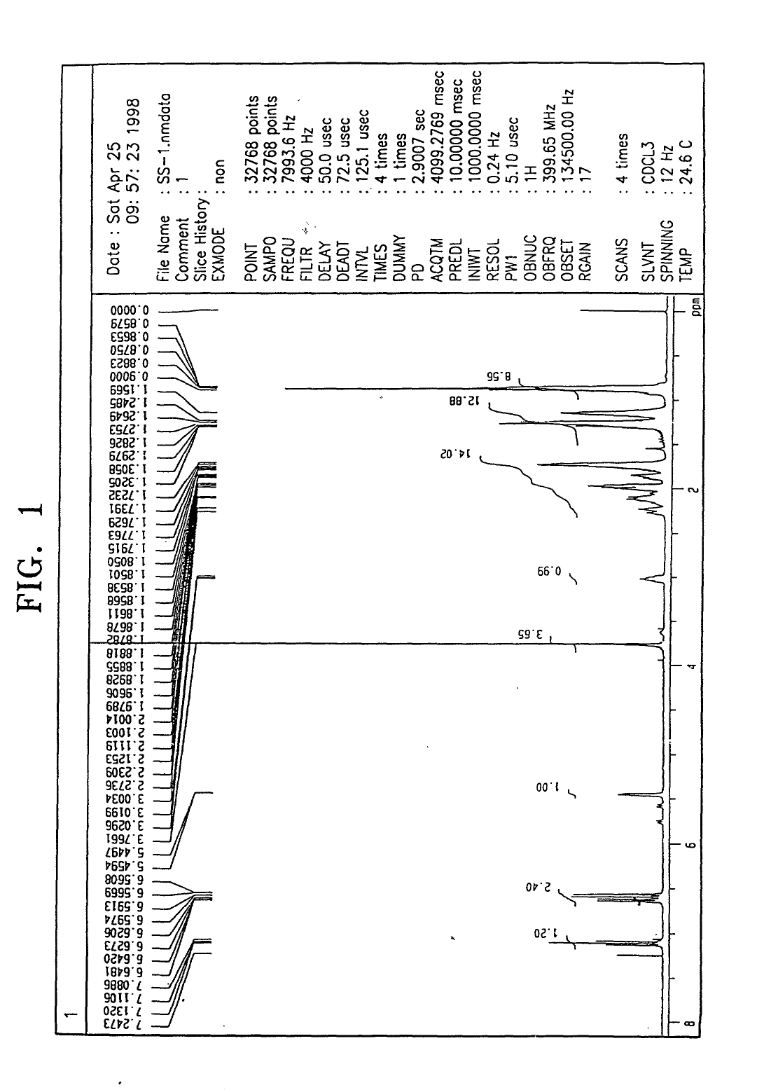

The same procedure of Example 1 was followed to synthesize trans-4-(4-methoxy-2-fluorophenyl)-1-[2-(trans-4-butylcyclohexyl)ethyl cyclohex-1-ene, with a yield of 58%. The NMR spectrum result of the product is shown in FIG. 1.

### Comparative Example

A liquid crystal composition containing 22% by weight 3-cyano-4-pentylbiphenyl, 20% by weight 4-(trans-4-pentylcyclohexyl)benzonitrile, 30% by weight 4-ethoxyphenyl ester of trans-4-butylcyclohexane carboxyl, and 28% by weight 4-butoxyphenyl ester of trans-4-butylcyclohexane carboxyl was prepared.

The liquid crystal composition was injected into cells to form a liquid crystal layer, so that a complete liquid crystal display was manufactured. The phase transitions with temperature in the liquid crystal layer were observed using a polarization microscope having a hot stage. The results are shown in Table 1.

### EXAMPLE 5

90% by weight the composition from Comparative Example 1, and 10% by weight the trans-4-(4-fluorophenyl)-1-[2-(trans-4-propyl cyclohexyl )ethyl]cyclohex-1-ene obtained in Example 1 were mixed to prepare a liquid crystal composition. The properties of the liquid crystal composition were observed in the same manner as in Comparative Example. The results are shown in Table 1.

### EXAMPLE 6

90% by weight the composition from Comparative Example 1, and 10% by weight the trans-4-(4-methoxy-2-fluorophenyl)-1-[2-(trans-4-butylcyclohexyl)ethyl cyclohex-1-ene obtained in Example 4 were mixed to prepare a liquid crystal composition. The properties of the liquid crystal composition were observed in the same manner as in Comparative Example. The results are shown in Table 1.

TABLE 1

| Example | T_{CrN}, C | T_{NI}, C | V_{10/20 C}, V | V_{90/20 C}, V | P_{10 20} |
|---|---|---|---|---|---|
| Comparative | 7 | 58 | 1.72 | 2.47 | 0.472 |
| Example | | | | | |
| Example 5 | 25 | 61 | 1.76 | 2.45 | 0.395 |
| Example 6 | 35 | 65 | 1.64 | — | 0.395 |

In Table 1, T_{Cr N} denotes the crystalline-nematic phase transition temperature, T_{N I} denotes the nematic-isotropic phase transition temperature, V_{10/20 C} denotes a threshold voltage at which 10% of the light is transmitted through the cells at 20 C, V_{90/20} C denotes a threshold voltage at which 90% of the light is transmitted through the cells at 20 C, and P_{10 20} denotes the steepness of an electro-optical curve.

Table 1 indicates that the liquid crystal compositions according to the present invention have a much lower T_{Cr N} and a higher T_{N I} compared to Comparative Example, and thus the nematic phase can be maintained over a wider temperature range. In summary, the liquid crystal composition according to the present invention has a low crystalline-nematic phase transition temperature, so that the working temperature range of a LCD device can be extended with a high-speed response.

While this invention has been particularly shown and described with reference to preferred embodiments thereof, it will be understood by those skilled in the art that various changes in form and details may be made thereto without departing from the spirit and scope of the invention as defined by the appended claims.

What is claimed is:

1. A liquid crystal compound of 1-[2-(trans-4-alkyl cyclohexyl)ethyl]-4-aryl cyclohexyl-1-ene having the formula: 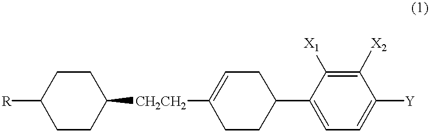
wherein R is CH₃(CH₂)ₙOₘ(CH₂)ₚ—; each of X₁ and X₂ is one of H, F and Cl; Y is F, Cl, —CN, —CF₃, —OCF₃ or an alkoxy group having 1 to 15 carbon atoms; each of n and p is an integer from 0 to 7; and m is 0 or 1.

2. The liquid crystal compound of claim 1, wherein in the formula (1), X₁ and X₂ are both H, Y is F, n=1, m=0 and p=0, to form trans-4-(4-fluorophenyl)-1-[2-(trans-4-propyl cyclohexyl)ethyl]cyclohex-1-ene.

3. The liquid crystal compound of claim 1, wherein in the formula, X₁ and X₂ are both H, Y is F, n=4, m=0 and p=0, to form trans-4-(4-fluorophenyl)-1-[2-(trans-4-pentylcyclohexyl)ethyl cyclohex-1-ene.

4. The liquid crystal compound of claim 1, wherein in the formula, X₁ is H, X₂ is F, Y is F, n=3, m=0 and p=0, to form trans-4-(4,3-di fluorophenyl)-1-[2-(trans-4-butylcyclohexyl)ethyl cyclohex-1-ene.

5. The liquid crystal compound of claim 1, wherein in the formula, X₁ is H, X₂ is F, Y is a methoxy group, n=3, M=0 and p=0, to form trans-4-(4-methoxy-2-fluorophenyl)-1-[2-(trans-4-butylcyclohexyl)ethyl cyclohex-1-ene.

6. A liquid crystal composition, comprising 1-[2-(trans-4-alkyl cyclohexyl)ethyl]-4-aryl cyclohexyl-1-ene having the formula: 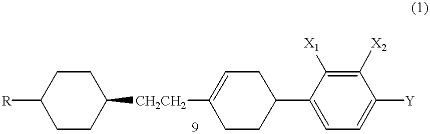
wherein R is CH₃(CH₂)ₙOₘ(CH₂)ₚ—; each of X₁ and X₂ is H, F or Cl; Y is F, Cl, —CN, —CF₃, —OCF₃ or an alkoxy group having 1 to 15 carbon atoms; each of n and p is an integer from 0 to 7; and m is 0 or 1.

7. A liquid crystal display, comprising a liquid crystal layer filled with a liquid crystal composition containing 1-[2-(trans-4-alkyl cyclohexyl)ethyl]-4-aryl cyclohexyl-1- ene having the formula 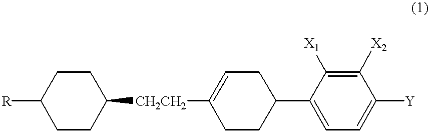
wherein R is CH₃(CH₂)ₙOₘ(CH₂)ₚ—; each of X₁ and X₂ is H, F or Cl; Y is F, Cl, —CN, —CF₃, —OCF₃ or an alkoxy group having 1 to 15 carbon atoms; each of n and p is an integer from 0 to 7; and m is 0 or 1.
